(19) United States
(12) Patent Application Publication
(10) Pub. No.: US 2010/0190997 A1
(43) Pub. Date: Jul. 29, 2010

(54) Process for the Preparation of Letrozole

(76) Inventors: Hussain Haider, Ahmedabad (IN); Kirtipalsinh Saijansinh Solanki, Ahmedabad (IN); Gautam Pal, Ahmedabad (IN); Manoj Kumar Singh, Ahmedabad (IN); Jay Shantilal Kothari, Ahmedabad (IN); Virendra Kumar Agarwal, Ahmedabad (IN)

Correspondence Address: LADAS & PARRY LLP, 26 WEST 61ST STREET, NEW YORK, NY, 10023, US

(21) Appl. No.: 12/083,665

(22) PCT Filed: Sep. 4, 2006
(86) PCT No.: PCT/IN2006/000337

§ 371 Date: Apr. 1, 2010

(30) Foreign Application Priority Data
Oct. 20, 2005 (IN) 1315-MUM-2005

(51) Int. Cl. C07D 249/08

(52) U.S. Cl. 548/269.4

(57) ABSTRACT

The present invention relates to the process for the preparation of Letrozole free from its regioisomer (7) and other impurities by selective extraction of desired intermediate (3) using suitable solvent and mixture of solvents.

### FIELD OF INVENTION

The present invention relates to a process for the preparation of letrozole free from its regioisomer and other impurities

### BACKGROUND OF THE INVENTION

Aromatase is an enzyme, which effects aromatisation of ring A in the metabolic formation of various steroid hormones. Various cancers, for example, breast cancer is dependent upon circulating steroid hormones, which have an aromatic ring A. Such cancers can be treated by removing the source of ring A aromatised steroid hormones, for example by the combination of oophorectomy and adrenalectomy. An alternative way of obtaining the same effect is by administering a chemical compound, which inhibits the aromatisation of the steroid ring A.

Letrozole is a non-steroidal antineoplastic, claimed to inhibit the aromatase (oestrogen synthase) activity. It is useful in the treatment of advanced breast cancer in postmenopausal women.

The growth of some cancers of the breast are stimulated or maintained by estrogens. Treatment of breast cancer thought to be hormonally responsive (i.e., estrogen and/or progesterone receptor positive or receptor unknown) has included a variety of efforts to decrease estrogen levels (ovariectomy, adrenalectomy, hypophysectomy) or inhibit estrogen effects (antiestrogens and progestational agents). These interventions lead to decreased tumor mass or delayed progression of tumor growth in some women.

In postmenopausal women, estrogens are mainly derived from the action of the aromatase enzyme, which converts adrenal androgens (primarily androstenedione and testosterone) to estrone and estradiol. The suppression of estrogen biosynthesis in peripheral tissues and in the cancer tissue itself can therefore be achieved by specifically inhibiting the aromatase enzyme.

Letrozole is a nonsteroidal competitive inhibitor of the aromatase enzyme system; it inhibits the conversion of androgens to estrogens. In adult tumor bearing females, Letrozole is as effective as ovariectomy in reducing uterine weight, elevating serum LH, and causing the regression of estrogen-dependent tumors. In contrast to ovariectomy, treatment with Letrozole does not lead to an increase in serum FSH. Letrozole selectively inhibits gonadal steroidogenesis but has no significant effect on adrenal mineralocorticoid or glucocorticoid synthesis.

Letrozole inhibits the aromatase enzyme by competitively binding to the heme of the cytochrome P450 subunit of the enzyme, resulting in a reduction of estrogen biosynthesis in all tissues. Treatment of women with Letrozole significantly lowers serum estrone, estradiol and estrone sulfate and has not been shown to significantly affect adrenal corticosteroid synthesis, aldosterone synthesis, or synthesis of thyroid hormones.

### DESCRIPTION OF PRIOR ART

Synthesis of Letrozole is reported in U.S. Pat. No. 4,978,672 and EP 236,940. In the above patents the synthesis of Letrozole starts with 4-bromomethylbenzonitrile (1), which undergoes condensation with 1,2,4-triazole (2) to form 4-[(1,2,4-triazol-1-yl)methyl]benzonitrile (3) as an intermediate. The compound of structural formula (3) is purified by column chromatography to remove 4-[(1,3,4-triazol-1-yl)methyl]benzonitrile (4) and followed by reaction with 4-fluorobenzonitrile (5) to afford Letrozole (6).

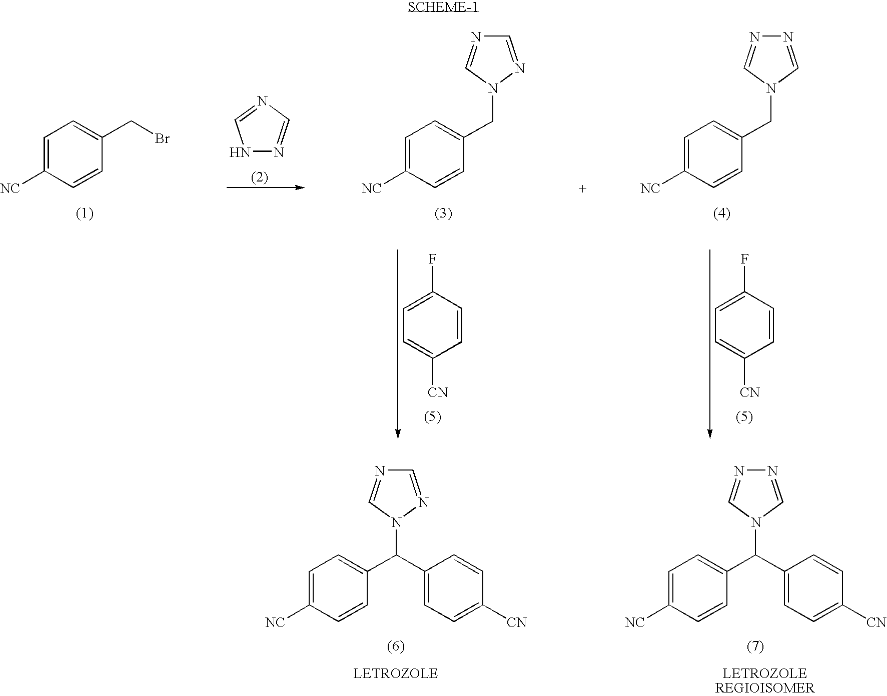

In the above process, the undesired intermediate 4-[(1,3,4-triazol-1-yl)methyl]benzonitrile (4) is formed during the course of the preparation 4-[(1,2,4-triazol-1-yl)methyl]benzonitrile (3) in 10% w/w to 30% w/w. The undesired impurity 4-[(1,3,4-triazol-1-yl)methyl] benzonitrile (4) present with 4-[(1,2,4-triazol-1-yl)methyl]benzonitrile (3), further on reaction with 4-fluorobenzonitrile (5) leads to the formation of another impurity 4-[1-(4-cyanophenyl)-1-(1,3,4-triazol-1-yl)methyl]benzonitrile (7) in approximately same ratio.

To control the formation of impurity of structural formula (7), it is required to make intermediate of structural formula (3) in its pure form.

The separation of desired compound from isomeric impurities is of great importance. In basic patents U.S. Pat. No. 4,978,672 and EP 236,940; chromatographic technique is used to isolate intermediate (3) from its mixture with regioisomer (4). Chromatography has its own limitations on commercial scale; it is an expensive and time consuming operation at plant scale, which also consumes lots of solvent and is hazardous for environment.

To overcome the above problems, purification of intermediate (3) is reported in PCT application WO 2005/047269 via the hydrochloride salt formation of the mixture of product (3) along with regioisomer (4). Selective crystallisation of regioisomer as hydrochloride using approximately 8.5 volume diisopropyl ether, filtering the resultant and then isolation of the intermediate (3) as pure product from the filtrate in approximately 60% overall yield. Finally washing the product with hexane or petroleum ether. In above PCT application, highly flammable solvents like diisopropyl ether, hexane and petroleum ether are used in process, which require high level of precautions and are never safe to handle on plant scale.

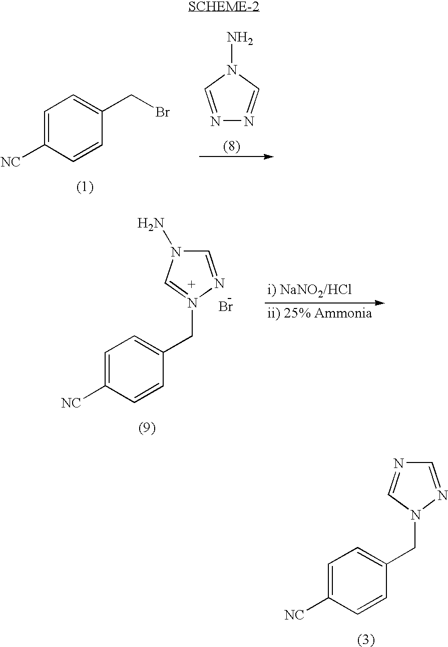

Another process reported in PCT application WO 2004/076409, discloses the different route to prepare the pure intermediate (3). The said patent discloses a reaction of 4-bromomethylbenzonitrile (1) with 4-amino-1,2,4-triazole (8) to give quaternary ammonium salt (9), which undergoes diazotisation reaction to give 4-[(1,2,4-triazol-1-yl)methyl]benzonitrile (3) in approximately 59% molar yield. The process is complicated and involves lengthy steps and tedious operations.

### OBJECTS OF THE INVENTION

It is therefore, an important object of the present invention to provide a process for the preparation of Letrozole which avoids the use of highly flammable solvents and is safe and smooth.

### SUMMARY OF THE INVENTION

To overcome the problems in the use of highly flammable solvents, complicated and lengthy steps and tedious operations; we have opted a simple and novel process for the purification of Letrozole intermediate (3), which is free from its regioisomer (4) and other related impurities.

Purification of intermediate (3) to remove its regioisomer (4) using crystallization method to achieve desired level of purity is unsuccessful. We have planned to go for extraction of intermediate (3) selectively from the mixture with regioisomer (4) in aqueous layer using a suitable solvent.

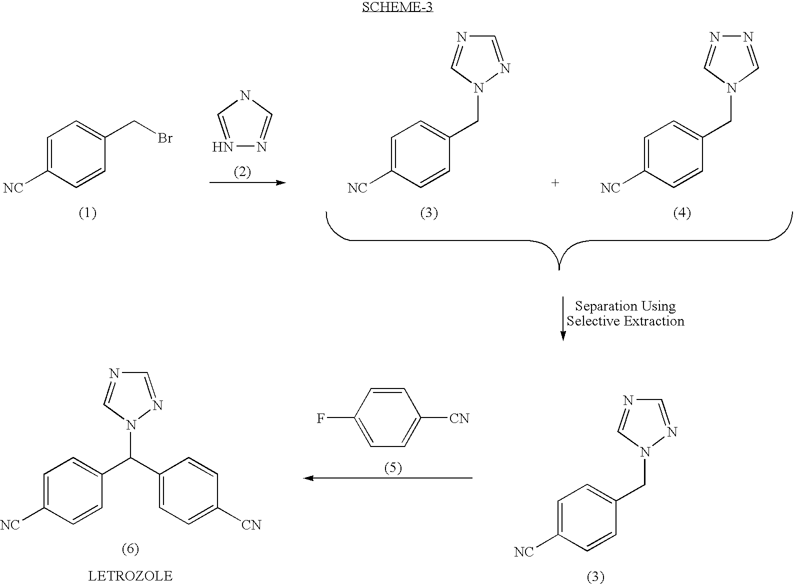

In order to obtain the pure Letrozole (6), we have planned to get intermediate (3) in its pure form and free from its regioisomer (4). For the said purpose, we have used solvent extraction method using suitable solvent system and selectively extract the desired intermediate 4-[(1,2,4-triazol-1-yl)methyl]benzonitrile (3), from a mixture with regioisomer 4-[(1,3,4-triazol-1-yl)methyl]benzonitrile (4). The control of the regioisomer at intermediate level leads its control at the final stage.

Therefore, in an embodiment, the present invention relates to Letrozole (6) with its regioisomer 4-[1-(4-cyanophenyl)-1-(1,3,4-triazol-1-yl)methyl]benzonitrile (7), preferably, less than 0.3% w/w, more preferably, less than 0.1% w/w and most preferably, below the quantitation limit.

In another feature, the present invention provides an improved process for the preparation of Letrozole with its regioisomer 4-[1-(4-cyanophenyl)-1-(1,3,4-triazol-1-yl)methyl]benzonitrile (7), preferably, less than 0.3% w/w, more preferably, less than 0.1% w/w and most preferably, below the quantitation limit.

In another feature, the present invention provides 4-[(1,2,4-triazol-1-yl)methyl]benzonitrile (3) with its regioisomer 4-[(1,3,4-triazol-1-yl)methyl]benzonitrile (4), preferably, less than 0.3% w/w, more preferably, less than 0.1% w/w and most preferably, below the quantitation limit.

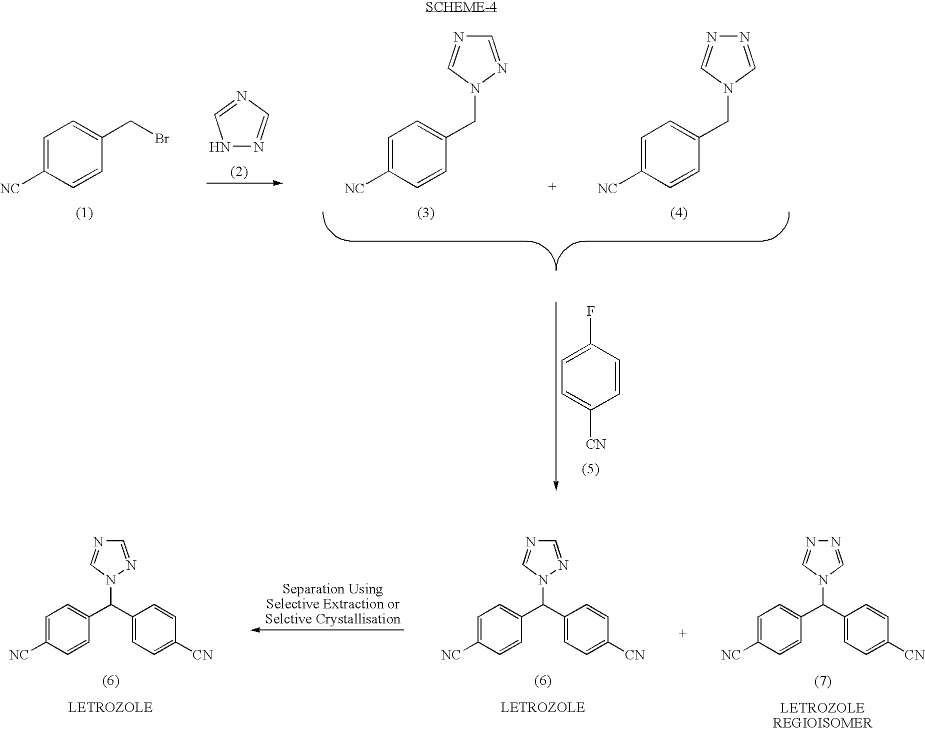

In yet another feature, the present invention provides an improved process for the preparation of 4-[(1,2,4-triazol-1-yl)methyl]benzonitrile (3) with its regioisomer 4-[(1,3,4-triazol-1-yl)methyl]benzonitrile (4), preferably, less than 0.3% w/w, more preferably, less than 0.1% w/w and most preferably, below the quantitation limit.

In order to obtain Letrozole (6) in purer form and free from its regioisomer (7) and other related impurities; intermediate 4-[(1,2,4-triazol-1-yl)methyl]benzonitrile (3) is to be prepared in its pure form, free from its regioisomer 4-[(1,3,4-triazol-1-yl)methyl]benzonitrile (4) and other related impurities. Thus, the main aspect of the present invention relates to the preparation of Letrozole (6) with its regioisomer (7) preferably less than 0.3%, more preferably less than 0.1% and most preferably below quantitation limit. For this purpose intermediate 4-[(1,2,4-triazol-1-yl)methyl]benzonitrile (3) is required to be of the same purity level. Another aspect of the present invention relates to the preparation of 4-[(1,2,4-triazol-1-yl)methyl]benzonitrile (3) with its regioisomer 4-[(1,3,4-triazol-1-yl)methyl]benzonitrile (4) preferably less than 0.3%, more preferably less than 0.1% and most preferably below quantitation limit.

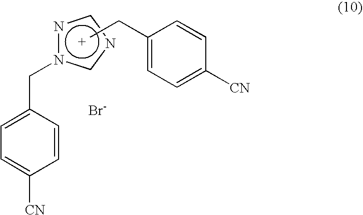

It has been also found that during the preparation of Letrozole intermediate 4-[(1,2,4-triazol-1-yl)methyl]benzonitrile (3) another impurity is formed, which is characterized as the quaternary salt (10). To control the formation of this quaternary salt, mole ratio of 1,2,4-triazole is optimized preferably from 1.5 mole to 4.4 mole equivalents and more preferably to 3.0 mole equivalents with respect to 4-bromomethylbenzonitrile (1). Thus, another aspect of the present invention relates to the preparation of Letrozole (6) with quaternary salt (10) preferably less than 0.1% and more preferably below quantitation limit.

Another important aspect of the present invention relates to the preparation of 4-[(1,2,4-triazol-1-yl)methyl]benzonitrile (3) in the pure form, free from its regioisomer 4-[(1,3,4-triazol-1-yl)methyl]benzonitrile (4). The purification of 4-[(1,2,4-triazol-1-yl)methyl]-benzonitrile (3) lakes place by its selective extraction from a mixture with its regioisomer 4-[(1,3,4-triazol-1-yl)methyl]benzonitrile (4) by using suitable solvents and/or mixture of solvents.

According to another aspect of the present invention Letrozole intermediate 4-[(1,2,4-triazol-1-yl)methyl]benzonitrile (3) is prepared with its regioisomer 4-[(1,3,4-triazol-1-yl)methyl]benzonitrile (4) less than 30% and followed by the preparation of Letrozole enriched with its regioisomer (7), which is removed by using crystallisation method using suitable solvent system.

### DETAILED DESCRIPTION OF INVENTION

The present invention relates, inter alfa, to an improved process of Letrozole with its regioisomer 4-[1-(4-cyanophenyl)-1-(1,3,4-triazol-1-yl)methyl]benzonitrile (7), preferably less than 0.3% w/w, more preferably less than 0.1% w/w and most preferably below quantitation limit and other impurities as discussed earlier below 0.1% w/w or below quantitation limits, which are arising due to impure intermediate 4-[(1,2,4-triazol-1-yl)methyl]benzonitrile (3) contaminated with regioisomer 4-[(1,3,4-triazol-1-yl)methyl]benzonitrile (4) and quaternary ammonium salt (10).

Particularly, the present invention relates to an improved and efficient process for the preparation of Letrozole with its regioisomer 4-[1-(4-cyanophenyl)-1-(1,3,4-triazol-1-yl)methyl]benzonitrile (7), preferably less than 0.3% w/w, more preferably less than 0.1% w/w and most preferably below quantitation limit and other impurities as discussed earlier preferably less than 0.1% w/w and most preferably below quantitation limits.

The impurities contaminating Letrozole are mainly due to impure intermediate 4-[(1,2,4-triazol-1-yl)methyl]benzonitrile (3) contaminated with regioisomer 4-[(1,3,4-triazol-1-yl)methyl]benzonitrile (4) and quaternary ammonium salt (10).

To obtain Letrozole in pure form with its regioisomer (7) and other related impurities to the said limits, intermediate 4-[(1,2,4-triazol-1-yl)methyl]benzonitrile (3) is required to be of the great purity free from its regioisomer (4) and other related impurities. The present invention relates to the removal of Letrozole intermediate regioisomer (4) from the reaction mixture. The regioisomer is removed by the selective extraction of intermediate (3) from the mixture of its regioisomer (4) using suitable solvents.

According to main embodiment of the present invention, the reaction of p-Cyanobenzylbromide (1) takes place with 1,2,4-Triazole (2) in Isopropanol in the presence of potassium carbonate at 60-65° C. After the completion of reaction, the reaction mixture is quenched in water and pH of the reaction mixture is adjusted to acidic by slowly addition of sufficient amount of cone. HCl. The reaction mixture on extraction with suitable solvent preferably hydrocarbons to extract desired product (3) selectively in organic layer. The aqueous layer is basified to pH 7.5 to 10, preferably 7.5-8.0, saturated with sodium chloride and again extracted using the same organic solvent to extract remaining intermediate (3). All the organic layers on mixing and washing with water remove any contamination of regioisomer (4) from the organic layer. Removal of solvent and crystallization of product from suitable solvent gives 4-[(1,2,4-triazol-1-yl)methyl]benzonitrile (3) in more than 99% purity with regioisomer below detection limits.

The pure intermediate 4-[(1,2,4-triazol-1-yl)methyl]benzonitrile (3) free from regioisomer (4) on reaction with p-Fluorobenzonitrile (5) in presence of base gives pure Letrozole free from its regioisomer (7) and other related impurities.

It has been observed that during the preparation of intermediate 4-[(1,2,4-triazol-1-yl)methyl]benzonitrile (3), another impurity is forming, which is isolated and characterized as the quaternary salt (10). The ¹H-NMR of the product in deuteriated DMSO shows, peaks at δ5.68 and 5.77 for two methylene protons, two multiplexes at δ7.64-7.74 and 7.90-7.96 for aryl protons and two singlets at δ9.40 and 10.44 for heterocyclic protons. 13C-NMR of the quaternary salt shows peaks at δ 49.99, 54.10, 111.59, 111.73, 118.42, 129.39, 129.81, 130.12, 132.70, 133.27, 137.47, 138.73, 143.62 and 145.19 supporting the impurity as quaternary salt (10).

According to another embodiment, the present invention relates to the preparation of Letrozole with quaternary salt preferably less than 0.1% and more preferably below quantitation limit. Formation of quaternary salt is controlled by optimization of the mole ratio of 1,2,4-triazole during the course of reaction. Mole ratio of 1,2,4-triazole is optimized preferably from 1.5 mole to 4.4 mole equivalent and more preferably to 3.0 mole equivalents with respect to 4-bromomethylbenzonitrile (1). Thus, according to another embodiment, the present invention relates to process for the preparation of Letrozole (6) with quaternary salt (10) preferably less than 0.1% and more preferably below quantitation limit.

The detailed schemes for the preparation of Letrozole are mentioned as Scheme-3 and Scheme-4. During the formation of 4-[(1,2,4-triazol-1-yl)methyl]benzonitrile (3), its regioisomer 4-[(1,3,4-triazol-1-yl)methyl]benzonitrile (4), is also formed in varying ratio from 10% w/w to 30% w/w, which is be removed by the selective crystallisation to the desired limit of purity using suitable solvent systems.

It is an embodiment of the present invention to go forward with crude mixture of 4-[(1,2,4-triazol-1-yl)methyl]benzonitrile (3), and its regioisomer 4-[(1,3,4-triazol-1-yl)-methyl]benzonitrile (4) to obtain a mixture of Letrozole and its regioisomer 4-[1-(4-cyanophenyl)-1-(1,3,4-triazol-1-yl)methyl]benzonitrile (7), and isolation of pure Letrozole using either selective extraction by the suitable solvent system or by fractional crystallisation.

According to another embodiment, the reaction of p-Cyanobenzylbromide (1) with 1,2,4-Triazole (2) on completion is extracted in a suitable solvent getting a 4-[(1,2,4-triazol-1-yl)methyl]benzonitrile (3) in crude form as a mixture with its regioisomer 4-[(1,3,4-triazol-1-yl)-methyl]benzonitrile (4) varying from 10% to 30% and other related impurities. The said reaction mixture further on reaction with p-Fluorobenzonitrile (5) in the presence of potassium tert-butoxide in THF gives crude Letrozole (6) after crystallization from isopropanol as a mixture with its regioisomer (7) ranging from 5% to 7%. The crude product on successive purification with suitable solvent, preferably, alcohols most preferably, methanol, gives desired purity of Letrozole (6), containing acceptable level of regioisomer (7).

However, if the starting material 4-bromomethylbenzonitrile (1) contains its raw material i.e. 4-toluonitrile as impurity, it also reacts with 4-fluorobenzonitrile and leads to the formation of 4,4′,4″-methylidenetrisbenzonitrile as another impurity in final product. So according to another embodiment, the present invention also relates to provide an improved process for preparation of Letrozole with impurity 4,4′,4″-methylidenetrisbenzonitrile (10), preferably less than 0.2% w/w, more, preferably, less than 0.1% w/w and most, preferably, below the quantitation limit.

The present invention also relates to the process for the preparation of Letrozole (6), with any known or unknown impurity preferably less than 0.1% w/w and more preferably below quantitation limit.

The present invention will now be described with reference to the following illustrative but non-limiting examples:

### Example 1

### Preparation of 4-[(1,2,4-triazol-1-yl)methy]benzonitrile (3)

In a 1.0 lit R. B. Flask fitted with a reflux condenser and a thermometer pocket, isopropanol (225 mL), p-cyanobenzylbromide (150 g), 1,2,4-triazole (160 g) and potassium carbonate (315 g) were charged to the reaction mixture at RT with stirring. The reaction mixture was heated to 60-65° C. for 1.0 hr. The progress of reaction was monitored over TLC for the completion of reaction. The reaction mixture was cooled down to RT and water (500 mL) was added to the reaction mixture and reaction mass was transferred to a three lit R. B. Flask containing water (1750 mL). Conc. HCl (420 mL) was added very slowly to the reaction mass to adjust pH 2-3. The reaction mixture was extracted from toluene (600 mL×3). Aqueous layer was basified to pH 8 using 20% sodium hydroxide solution (60 mL). The aqueous layer was saturated with sodium chloride (660 g) and again extracted from fresh toluene (600 mL×3). All the six toluene extracts were combined and washed with water (720 mL×2). Both the aqueous washings were combined and extracted with toluene (290 mL×2). Both the toluene extracts were combined and washed with water (110 mL×2). The entire toluene layers were collected and distilled completely under vacuum at 60° C. To the residue cyclohexane (100 mL) was added and distilled completely to get solid product, which was recrystallised from a mixture of isopropanol and cyclohexane to give 90 gm of pure 4-[(1,2,4-triazol-1-yl)methyl]benzonitrile (3) free from regioisomer. Melting Point: 78-80° C. HPLC Purity>99%. Regioisomer˜Not Detected.

### Example 2

### Preparation of 4-[1-(4-cyanophenyl)-1-(1,2,4-triazol-1-yl)methyl]benzonitrile (6)

In a one liter three neck flask, equipped with thermometer pocket, mechanical stirrer and a guard tube, THF (500 mL) was charged at room temperature. Potassium tert-butoxide (122 gm) was added in small portions in 30 minutes. The solution was cooled to −15° C. and a solution of 4-[(1,2,4-triazol-1-yl)methyl]benzonitrile (100 g), and p-fluorobenzonitrile (86 g) in THF (500 mL) was added very slowly to the reaction mixture in 4-5 hrs. The reaction mixture was stirred at same temperature for 3 hrs. Progress of the reaction was monitored on TLC. Dichloromethane (1000 mL) was added to the reaction mixture followed by the addition of acetic acid (65 g). Reaction mixture was added to another flask containing water (1100 ml). pH of the reaction mixture was adjusted to 7-8 by addition of 5% sodium bicarbonate solution (900 mL). Dichloromethane layer was separated out and again washed with water (1000 mL). Dichloromethane layer separated, filtered through hyflow bed and distilled at a temperature below 50° C. The residue obtained was crystallized by the addition of Isopropanol. (100 mL) to get the crude product (82 g), which was recrystallised from methanol (160 mL) to get pure Letrozole (74 gm). HPLC Purity>99.8%. Regiosiomer: Not Detected.

### Example 3

### 4-[(1,2,4-triazol-1-yl)methyl]benzonitrile (3) with a mixture of 4-[(1,3,4-triazol-1-yl)methyl]benzonitrile (4)

To a 250 mL three neck R. B. Flask fitted with a reflux condenser and a thermometer pocket, isopropanol (37.5 mL), p-cyanobenzylbromide (25 g), 1,2,4-triazole (26.4 g) and potassium carbonate (52.8 g) were charged to the reaction mixture at RT with stirring. The reaction mixture was heated to 60-65° C. for 1.0 hr. The progress of reaction was monitored over TLC for the absence of p-cyanobenzylbromide. After completion, the reaction mixture was cooled down to RT and water (100 mL) was added to the reaction mixture and reaction mass was transferred to a one lit R. B. Flask containing water (275 mL). Conc. HCl (50 mL) was added very slowly to the reaction mass to adjust pH 7-8. The reaction mixture was extracted from dichloromethane (250 mL). Dichloromethane layer was distilled out at 50° C. giving 21.0 gm viscous oily residue. The residue is crystallized from a mixture of IPA: Cyclohexane (1:10) to give 18 g of 4-[(1,2,4-triazol-1-yl)methyl]benzonitrile (3) with a mixture of its regioisomer (4). HPLC Purity˜85%; Regioisomer˜13%.

### Example 4

### 4-[1-(4-cyanophenyl)-1-(1,2,4-triazol-1-yl)methyl]benzonitrile (6) & 4-[1-(4-cyanophenyl)-1-(1,3,4-triazol-1-yl)methyl]benzonitrile (7)

In a 250 mL three neck flask, equipped with thermometer pocket, mechanical stirrer and a guard tube, THF (50 mL) was charged at room temperature. Potassium tert-butoxide (12.3 gm) is added in small portions in 30 minutes. The solution was cooled to −15° C. and a solution of product from example—3 (10 g) and p-fluorobenzonitrile (8.5 g) in THF (50 mL) is added very slowly to the reaction mixture in 4-5 hrs. Stir the reaction mixture at same temperature for 3 hrs. Progress of the reaction is monitored on TLC. Dichloromethane (200 mL) is added to the reaction mixture followed by the addition of acetic acid (7 g). Reaction mixture is added to another flask containing water (220 ml). pH of the reaction mixture is adjusted to 7-8 by addition of 5% sodium bicarbonate solution (180 mL). Dichloromethane layer is washed with water (200 mL), separated, filtered through hyflow bed and distilled at a temperature below 50° C. The residue obtained was crystallized from Isopropanol (20 mL) to get the solid product (4.9 g). HPLC Purity: 89.6%. Regioisomer: 7.41%.

### Example 5

### Removal of Regioisomer (7) from Letrozole (6)

To a 250 mL R. B. Flask crude Letrozole (4.5 g) was charged in methanol (115 mL) and heated to 60° C. to dissolve completely and get clear solution. Methanol (approx. 100 mL) was distilled out and the solution was cooled to 25-30° C., and was stirred for 2 hrs at this temperature. Solid product was filtered and washed with methanol (10 mL×2) to get solid product, which was dried in vacuum to get 3.5 gm of product.

HPLC Purity: 96.33%, Regioisomer: 3.4%

Using the same purification method, desired purity of Letrozole had been achieved containing acceptable level of regioisomer (7).

| | Raw | Wt./ | | Yield | Compound | Regioisomer |
|---|---|---|---|---|---|---|
| | Materials | Vol. | | (g) | (6) | (7) |
| Ist | Letrozole-1 | 4.5 | g | 77.8% | 96.33% | 3.4% |
| Purifi- | Methanol | 142 | mL | (3.5 g) | | |
| cation | | | | | | |
| II^{nd} | Letrozole-2 | 3.0 | g | 93.3% | 99.76% | 1.08% |
| Purifi- | Methanol | 85 | mL | (2.8 g) | | |
| cation | | | | | | |
| III^{rd} | Letrozole-3 | 2.8 | g | 78.6% | 99.29% | 0.57% |
| Purifi- | Methanol | 80 | mL | (2.2 g) | | |
| cation | | | | | | |

Following the above purification from methanol repeatedly, the Letrozole may be prepared with the desired limit of its regioisomer (7).

1. A process for the preparation of Letrozole comprising;
a) reacting 4-bromomethylbenzonitrile of structural formula (1) 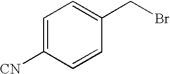
with a 1,2,4-triazole of structural formula (2) 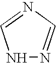
to obtain a crude intermediate of structural formula (3) 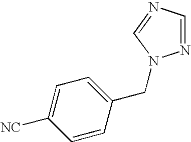
containing up to approximately 0% to 30% w/w of regioisomer of structural formula (4) in the aqueous solution; 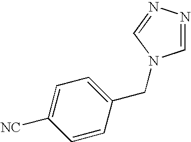
b) isolating pure intermediate (3) from its mixture with undesired regioisomer (4) by using selective extraction in one or more solvents of the kind such as herein described, keeping regioisomer (4) in aqueous layer;
c) reacting pure intermediate of structural formula (3) with 4-fluorobenzonitrile (5); 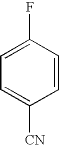
d) isolating Letrozole from the reaction mixture and
e) purifying Letrozole from a suitable solvent.

2. A process as claimed in claim 1 wherein Letrozole (6) 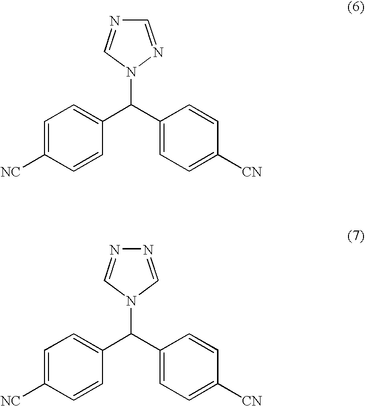
is prepared with its regioisomer (7) less than 0.3% w/w, more preferably, less than 0.1% and most preferably, below quantitation limit.

3. A process as claimed in claim 1 wherein the amount of 1,2,4-triazole (2) is in the range of more than 1.5 mole equivalent and less than 4.4 mole equivalent with respect to 4-bromomethylbenzoniπle of structural formula (1).

4. A process as claimed in claim 1, wherein the solvent used for selective extraction is a hydrocarbon solvent.

5. A process as claimed in claim 1, wherein Letrozole is purified in alcoholic solvents preferably, C1-C4 alcoholic solvents and more preferably, methanol.

6. A process as claimed in claim 1, wherein Letrozole is prepared with quaternary ammonium salt (10), 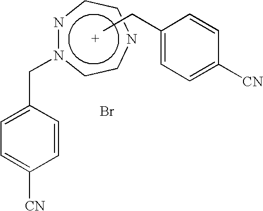
preferably less than 0.1% w/w and more preferably below quantitation limits.

7. A process as claimed in claim 1, wherein Letrozole is prepared with any other impurity, preferably less than 0.1% w/w and more preferably below quantitation limit.

8. A process as claimed in claim 1, wherein the intermediate (3) is prepared with its regioisomer (4) less than 0.3% w/w, more preferably less than 0.1% and most preferably blew quantitation limit.

9. A process as claimed in claim 8 wherein said intermediate is 4-[(1,2,4-triazol-1-yl)methyl]benzonitrile (3) and is prepared with said quaternary salt (10) preferably less than 0.1% w/w and most preferably below quantitation limit and other related impurities preferably less than 0.1% w/w and most preferably below quantitation limits.

10. A process as claimed in claim 9 wherein said 4-[(1,2,4-triazol-1-yl)methyl]benzonitrile (3) is prepared with its regioisomer 4-[(1,3,4-triazol-1-yl)methyl]benzonitrile (4), preferably less than 0.3% w/w, more preferably less than 0.1% w/w and most preferably below quantitation limit.

11. A process as claimed in claim 1, wherein said Letrozole is prepared with impurity 4,4′,4″-methylidenetrisbenzonitrile (10), preferably less than 0.2% w/w, more preferably less than 0.1% w/w and most preferably below quantitation limit.

12. A process for the preparation of Letrozole comprising;
a) reacting crude intermediate of structural formula (3) 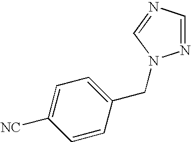
containing up to 30% w/w of regioisomer of structural formula (4) 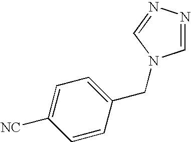
with 4-fluorobenzonitrile (5);
b) isolating crude Letrozole containing its regioisomer up to 30% w/w; and
c) purifying Letrozole by selective crystallization method using suitable solvent system.
